(19) United States
(12) Patent Application Publication
(10) Pub. No.: US 2023/0149187 A1
(43) Pub. Date: May 18, 2023

(54) ACTUATABLE PROSTHESIS DEVICE, ELECTRONIC DATA PROCESSING DEVICE, AND METHOD FOR ACTUATING A PROSTHESIS DEVICE

(71) Applicant: OTTO BOCK HEALTHCARE PRODUCTS GMBH, Wien (AT)

(72) Inventors: Sebastian AMSÜSS, Wien (AT); Carina Doris BALL, Wien (AT); Patrick MAYRHOFER, Wien (AT); Luis SAGMEISTER, Pitten (AT); Johannes STEININGER, Kleinbaumgarten (AT)

(73) Assignee: OTTO BOCK HEALTHCARE PRODUCTS GMBH, Wien (AT)

(21) Appl. No.: 17/917,098

(22) PCT Filed: Apr. 1, 2021
(86) PCT No.: PCT/EP2021/058674

§ 371 Date: Oct. 5, 2022

(30) Foreign Application Priority Data
Apr. 6, 2020 (DE) 10 2020 109 509.0

(51) Int. Cl. A61F 2/72; G16H 40/67

(52) U.S. Cl. CPC A61F 2/72; G16H 40/67; A61F 2002/704

(57) ABSTRACT

An actuatable prosthesis device, comprising at least one drive, an electronic data processing device, and at least one sensor assembly for arranging on a body part of a prosthesis wearer. The at least one sensor assembly is designed to detect body signal patterns, and the electronic data processing device is designed to actuate the at least one drive on the basis of a detected body signal pattern such that the prosthesis devicecarries out a prosthesis movement assigned to the body signal pattern wherein at least two sets of body signal patterns and prosthesis movements assigned to each body signal pattern are stored on the electronic data processing device, and at least one trigger signal which switches over between the sets is assigned to the sets.

### CROSS REFERENCE TO RELATED APPLICATIONS

This is a national phase application of International Application No. PCT/EP2021/058674, filed 01 Apr. 2021, which claims the benefit of German Patent Application No. 10 2020 109 509.0, filed 06 Apr. 2020, the disclosures of which are incorporated herein, in their entireties, by this reference.

### TECHNICAL FIELD

The invention relates to an actuatable prosthesis device, having at least one drive, an electronic data processing device, and at least one sensor assembly for arranging on a body part of a prosthesis wearer, wherein the at least one sensor assembly is designed to detect body signal patterns, and the electronic data processing device is designed to actuate the at least one drive depending on a detected body signal pattern in such a way that the prosthesis device carries out a prosthesis movement assigned to the body signal pattern.

According to another aspect, the invention also relates to an electronic data processing device for such a prosthesis device.

According to a third aspect, the invention relates to a method for actuating such a prosthesis device.

### BACKGROUND

Actuatable prostheses are, for example, prostheses of the upper or lower extremities. Prosthesis devices are also prosthetic parts, such as a prosthetic hand or a prosthetic foot. Modern prosthesis devices are usually controlled by means of an electronic data processing device. For this purpose, the prosthesis device is assigned at least one drive, which can be activated via the electronic data processing device. In order to be able to carry out the most targeted movement possible and to be able to trigger it as intuitively and easily as possible by a prosthesis wearer, such an actuatable prosthesis device usually has at least one sensor assembly. This is often arranged on a part of the body of the prosthesis wearer to which the prosthesis device is assigned.

Using the at least one sensor assembly, for example, it is then possible to detect the intentional body signals of the prosthesis wearer and to route them to the electronic data processing device. In this device, individual prosthesis movements are then assigned to the body signals, so that the electronic data processing device can control at least one drive in such a way that the prosthesis device performs the prosthesis movement assigned to the body signal. Such body signals are, for example, the contraction, or co-contractions, of specific individual muscles or muscle groups at a specific intensity and/or frequency.

It is also known to detect so-called body signal patterns by means of the at least one sensor assembly. Such body signal patterns are complex signal patterns, such as sequences of muscle contractions of the amputation stump. These can be selectively practiced by a prosthesis wearer and can be oriented, for example, to a movement pattern that the prosthesis wearer used in the intact state to move the limb that is now supplied with the prosthesis. Especially in patients whose amputation took place recently, a good memory of the necessary muscle contractions remains. For example, radial-amputated patients can perform a contraction of the forearm muscles that was associated with a gripping movement of the hand in the intact state. These body signal patterns are then preferably detected by means of a sensor assembly, stored in the electronic data processing device and/or a storage medium assigned to it, and assigned to a specific prosthesis movement, in this example, in particular to such a gripping movement. If a certain movement or muscle contraction pattern is performed and detected by the sensor assembly as a certain body signal pattern, a command will be transmitted to the at least one drive via a controller stored in the electronic data processing device to perform a specific displacement or pivoting of a prosthesis component over a specific path in a specific time.

However, the problem here is that due to their complexity, only a limited number of such body signal patterns can be generated by a prosthesis wearer. This limits the number of assignable prosthesis movements accordingly, which prevents the prosthesis device from simulating the replaced body part as realistically as possible. Even very good and experienced prosthesis wearers can rarely generate more than eight body signal patterns reproducibly.

### SUMMARY

Against this background, it is the object of the present invention to propose a prosthesis device that can be actuated as simply as possible, with which a larger number of prosthesis movements are possible.

The invention achieves this object by means of an actuatable prosthesis device having the features of the main claim, by an electronic data processing device for such a prosthesis device, and a method for actuating such a prosthesis device. Advantageous embodiments and developments of the invention are disclosed in the subclaims, the description and the figures.

The actuatable prosthesis device, having at least one drive, an electronic data processing device and at least one sensor assembly for positioning on a part of the body of a prosthesis wearer, wherein the at least one sensor assembly is configured to detect body signal patterns and the electronic data processing device is configured to actuate the at least one drive depending on a detected body signal pattern, in such a way that the prosthesis device performs a prosthesis movement assigned to the body signal pattern, provides that at least two sets of body signal patterns and prosthesis movements assigned to them are stored in the electronic data processing device, and that at least one trigger signal that switches between the sets is assigned to the sets.

The fact that at least two sets of body signal patterns and prosthesis movements assigned to them are stored in the electronic data processing device means it is a simple matter to increase the number of prosthesis movements that can be performed by the actuatable prosthesis device.

In the simplest case, the electronic data processing device contains exactly two sets and exactly one trigger signal. In order to carry out a specific prosthesis movement, the prosthesis wearer performs a movement, in particular a muscle contraction pattern, which is detected by the sensor assembly and is recognized by the electronic data processing device as a specific body signal pattern. The electronic data processing device then first checks which of the two sets is selected and activates the drive according to the prosthesis movement assigned to the body signal pattern in the selected set. For example, to switch over from the first set to the second set, the trigger signal must be initiated a first time. To switch back from the second set to the first set, the trigger signal is initiated again. If the prosthesis wearer has selected the desired set using the trigger signal, he/she performs the movement or the muscle contraction pattern that corresponds to the body signal pattern for the desired prosthesis movement. Such sequential switching offers the advantage that the prosthesis wearer only has to learn and/or memorize one trigger signal.

According to a development, each set is assigned a different trigger signal, which then accordingly codes just for this set. For example, a first trigger signal can be assigned to a first set, a second trigger signal to a second set, and a third trigger signal to a third set. Initiating the respective trigger signal switches directly over to the associated set. This is advantageous in the case of two sets, for example, because the prosthesis wearer does not need to know which set is currently selected in order to select a particular set. This embodiment is particularly advantageous when more than two sets are available, since in particular a complex sequential switching process is not required.

The prosthesis device in one development has a display device that displays the currently selected set. For example, this is a display or one or more LEDs.

One of the sets is preferably specified as the default set. For example, if a trigger signal initiated by the prosthesis wearer was not used to switch from another set to this set, then the default set is selected automatically. For example, this takes place automatically after a pre-defined time period of, for example, at least 10 seconds, in particular at least 30 seconds. Preferably, this time period can be set individually. In this way, it is not necessary for the prosthesis wearer to remember the currently selected set, or for a display device to indicate the currently selected set.

If the prosthesis wearer has not made the prosthesis device perform a movement for a period of time, the wearer knows that he/she will always be in the first set. However, it is possible that in addition to such a time-controlled switchover to the default set, a display device for the currently selected set is available. Alternatively, the default set is initially only selected when switching on, and if a trigger signal is subsequently generated and the device is switched over from the default set to a first or second set, these can also remain in the selected state.

Preferably at least one body signal pattern contained in the sets is identical, wherein the at least one identical body signal pattern contained in the sets is assigned different prosthesis movements.

The fact that at least one body signal pattern contained in the sets is identical ensures that this at least one body signal pattern is duplicated, i.e. the same body signal pattern is encoded for more than one prosthesis movement. By selecting a specific set using the at least one trigger signal, the prosthesis movement to be performed via the specific body signal pattern can be selected accordingly.

It is also possible that more than one body signal pattern contained in the sets is identical. For example, two, three, four, five, six, seven or eight of the body signal patterns contained in the sets can be identical.

Preferably, each set contains the identical body signal patterns. In other words, each set contains exactly the same body signal patterns, with each body signal pattern in the different sets preferably having different prosthesis movements assigned to it. For example, in a first set, the prosthesis movements 1 to 4 are assigned to the body signal patterns 1 to 4. In the second set, the prosthesis movements 5 to 8 are then assigned to the body signal patterns 1 to 4. In this way, it is possible to perform eight different prosthesis movements with only four body signal patterns.

In a development, the same prosthesis movements are assigned to one or more body signal patterns in more than one set, preferably in every set. For example, in a first set, the prosthesis movements 1 to 4 are thus assigned to the body signal patterns 1 to 4. In the second set, the body signal pattern 1 is again assigned the prosthesis movement 1, but the body signal patterns 2 to 4 are assigned prosthesis movements 5 to 7. This is particularly advantageous when a prosthesis movement, in the example the prosthesis movement 1, is a particularly frequently performed prosthesis movement. This can then be triggered with the same body signal pattern, regardless of which set is currently to be used. This avoids unnecessarily frequent switching over by means of the at least one trigger signal, and increases the convenience for the user.

According to a development of the invention, at least three sets, more preferably at least four sets, are stored in the electronic data processing device so that the number of prosthesis movements is greatly increased without the requirements on the prosthesis wearer, namely to learn further body signal patterns, needing to be increased.

The at least one trigger signal is preferably a specified body signal that differs from the body signal patterns stored in the sets. Therefore, it is preferable that the at least one trigger signal is not an independent body signal pattern, but a body signal such as a co-contraction of a certain muscle or muscle group. This is particularly advantageous in order not to assign the at least one trigger signal to one of the few available body signal patterns, but rather to keep it free for one or more prosthesis movements. If different trigger signals are assigned to a specific set, for example, the respective trigger signals can belong to the same category, i.e. they can all be different co-contractions, for example. However, it is equally possible that the different trigger signals belong to different categories, such as co-contractions, contraction patterns, contraction duration, and/or contraction intensity. Preferably, each set comprises at least four body signal patterns, in particular at least six body signal patterns.

The number of body signal patterns per set can be determined, for example, depending on the level of experience of a prosthesis wearer for whom the particular prosthesis device is intended. For inexperienced and untrained prosthesis wearers, for example, it may be advantageous to provide only exactly two, exactly three or exactly four body signal patterns per set. For some, in particular more experienced wearers, however, it may be advantageous if exactly five, exactly six, exactly seven or exactly eight body signal patterns are stored per set. Preferably, each set has no more than twelve, particularly preferably no more than 10 body signal patterns, as this would not be manageable with the necessary precision and discrimination for the vast majority of prosthesis users.

Preferably, the at least one sensor assembly has a plurality of electrode pairs for detecting the body signal patterns and, in particular, one electrode pair for detecting the at least one trigger signal.

The at least one sensor assembly preferably has a plurality of electrode pairs, for example at least two, particularly preferably at least four, in particular exactly four electrode pairs, for detecting the body signal patterns. It is possible that a further pair of electrodes is present for detecting the at least one trigger signal. However, it is also possible that one of the electrode pairs used to detect the body signal patterns also acts as the electrode pair used to detect the at least one trigger signal. The sensor assembly or the electrode pair used to detect the at least one trigger signal may also be arranged in particular on a limb or a body part that is not functionally related to the prosthesis or prosthesis component.

The electrodes of the at least one sensor assembly are preferably arranged evenly spaced or substantially evenly spaced around the limb, the limb stump or the amputation stump, for example with six electrodes at intervals of 60° or approximately 60° to each other, with eight electrodes at intervals of 45° or approximately 45° to each other, and with four electrodes at intervals of 90° or approximately 90° to each other.

The number of electrodes or electrode pairs required depends on the complexity of each prosthesis device and the movements it must perform. For complex prosthetic treatments, to which this invention relates in particular, for example with a driven prosthetic elbow joint, a prosthetic hand with individually movable prosthetic fingers and/or a flectional and/or rotatable wrist joint, more than eight pairs of electrodes are sometimes required.

The type of treatment and the number of electrode pairs also depend on the wearer’s ability to generate certain complex body signal patterns.

Preferably, the sets can be at least partially changed using an input device. For this purpose, a computer can be connected to the electronic data processing device, wirelessly or via a cable, and the sets stored in it can be changed. According to a development, this is possible via an app which is stored, for example, on a smartphone of the respective prosthesis wearer or an orthopedic technician.

The fact that the sets are variable means, for example, that a number of body signal patterns stored per set is variable and/or the stored prosthesis movements and/or the assignment of a specific prosthesis movement to a specific body signal pattern are variable. In addition or alternatively, the body signal patterns themselves, i.e. in particular which muscles or muscle groups must be contracted and in what way for a particular body signal pattern, or the specifications and parameters for them, can be adjusted or changed.

By using the at least one sensor assembly, it is preferably possible to detect electromyographic signals, which can then be evaluated with regard to their signal quality. Signal quality is understood to include the separability of the signals, in particular the shape or waveform of the signal or the location where the signal is detected. Furthermore, the repeatability of the signal is also a quality criterion. A high signal quality is achieved if the signal always looks the same, thus if a patient can always generate the same signal. The sustainability of the signal is also relevant to the signal quality. If the signal can be generated and detected for a longer period of time, this is better than a short-duration signal, which might in some cases be interpreted as an interfering variable and ignored. To this end, the patient with the at least one sensor assembly attached is prompted to perform muscle activities, such as performing the task that the prosthesis device is designed to perform, such as opening the hand, closing the hand, turning the wrist, or flexing the hand. Of course, this task cannot be performed directly by the patients, as the limb replaced by the prosthesis device is missing, but the remaining muscles can be partially activated as they were before the loss of the limb. This generates electrical potentials that are detected by the at least one sensor assembly and transmitted to the electronic data processing device. For example, it is possible to determine whether a specific movement or a specific muscle contraction pattern is suitable for use as a body signal pattern based on the signal duration, signal intensity, edge gradient or even a signal frequency in the case of muscle contractions.

It is preferably possible to use the input device, in particular the app, to indicate to a prosthetic wearer which sensor assembly is currently detecting which signal. In particular, the signal quality of the signal is also displayed visually. This allows the prosthetic wearer to perform certain muscle contraction patterns and to be displayed using the input device, how much of this the at least one sensor assembly has recognized. This makes it possible, in particular, for the prosthesis wearer to practice the execution of the body signal patterns or for the body signal patterns to be adapted to the actual sensor values achieved.

According to another aspect, the invention achieves the object addressed by an electronic data processing device for such a prosthesis device, wherein at least two sets of body signal patterns and prosthesis movements assigned to them are stored in the electronic data processing device, and that at least one trigger signal that switches between the sets is assigned to the sets.

According to a third aspect, the invention achieves the object addressed by a method for actuating a prosthesis device, having the steps: a) selecting a set to be applied from the at least two sets stored in the electronic data processing device by issuing the at least one trigger signal, b) detecting a body signal pattern of the prosthesis wearer using the at least one sensor assembly, and c) actuating the prosthesis device by means of the at least one drive depending on the detected body signal pattern and the selected set, so that the prosthesis device performs the prosthesis movement assigned to the detected body signal pattern in the selected set.

According to a development, step c) comprises the following steps: routing a sensor signal coding for the detected body signal pattern from the sensor assembly to the electronic data processing device, evaluating the sensor signal in the electronic data processing device, determining a prosthesis movement to be performed, depending on the sensor signal and the selected set and generating a control signal that codes for the prosthesis movement to be performed, routing the control signal to the at least one drive of the prosthesis device, and operating the at least one drive depending on the control signal so that the prosthesis device performs the prosthesis movement to be performed.

The sensor signal or the sensor signals and the control signal are routed to the electronic data processing device, for example, by means of a data cable or wirelessly.

### BRIEF DESCRIPTION OF THE DRAWINGS

Hereafter the invention is explained in more detail by reference to the accompanying drawings, in which

FIG. 1 shows a visualization of an embodiment of the electronic data processing device with body signal patterns stored therein and prosthesis movements assigned to them,

FIG. 2 shows a schematic flowchart of an embodiment of the method,

FIG. 3 shows a schematic representation of an embodiment of the actuatable prosthesis device in the form of a prosthesis for an upper extremity, and

FIG. 4 shows a schematic representation of an embodiment of the actuatable prosthesis device in the form of a prosthesis for a lower extremity.

### DETAILED DESCRIPTION

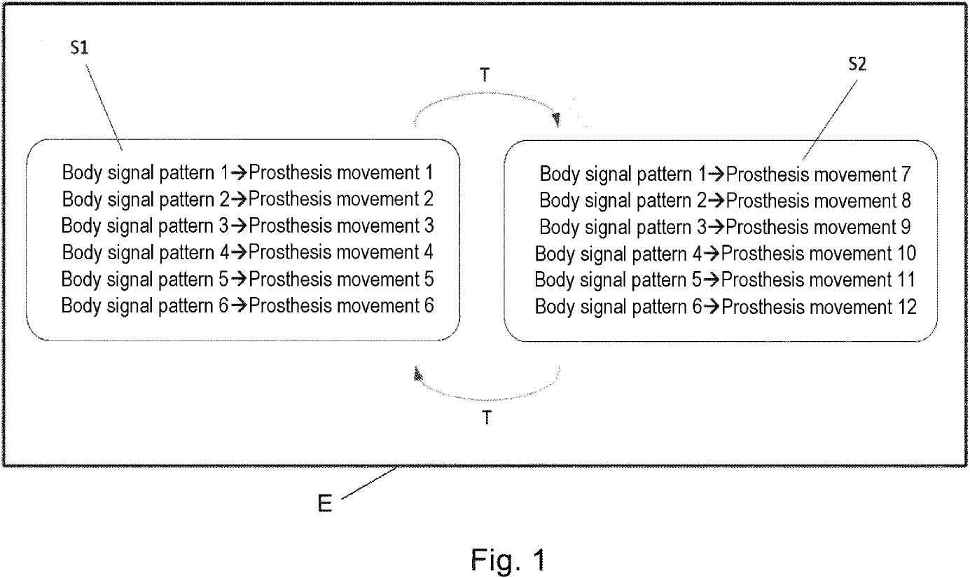

FIG. 1 indicates an electronic data processing device E, in which a first set S1 and a second set S2 are stored. The sets S1, S2 each have six body signal patterns. The body signal patterns in both sets S1 and S2 are completely identical, namely the body signal patterns 1 to 6 in each case. Each body signal pattern is assigned a prosthesis movement. The prosthesis movements 1 to 6 are assigned to the body signal patterns 1 to 6 in the first set S1. In the second set S2, different prosthesis movements, namely the prosthesis movements 7 to 12, are then assigned to the same body signal patterns 1 to 6.

This division enables twelve different prosthesis movements to be triggered with only six different body signal patterns.

In the embodiment shown in FIG. 1, only one trigger signal T is present. In order to switch between the sets S1, S2, the prosthesis wearer must implement or generate the trigger signal T. This is, for example, a co-contraction of a certain muscle or a certain muscle group. This trigger signal T is then used to switch between the sets S1, S2. In accordance with another embodiment, the first set S1 is assigned a first trigger signal T1 and the second set S2 is assigned a second trigger signal T2. In this case the first trigger signal T1 differs from the second trigger signal T2. Issuing or generating the first trigger signal T1 always switches over to the first set S1, and issuing or executing the second trigger signal T2 always switches to the second set S2.

One of the two sets S1 or S2 is preferably assigned an additional, time-dependent switchover signal. For example, the electronic data processing device E automatically switches over from set S2 to set S1 after 10 seconds, 20 seconds or 30 seconds, if this has not been carried out by the wearer of the prosthesis device by initiating the trigger signal T. In this example, set S1 then forms the default set.

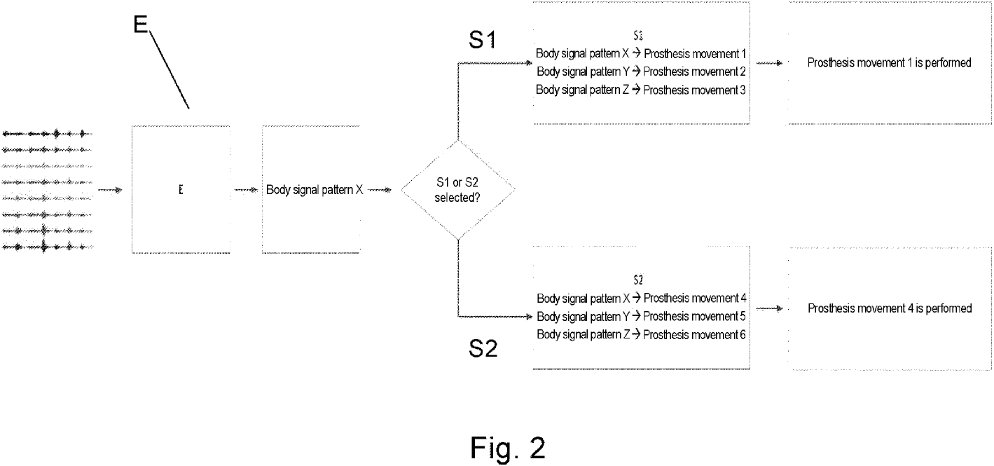

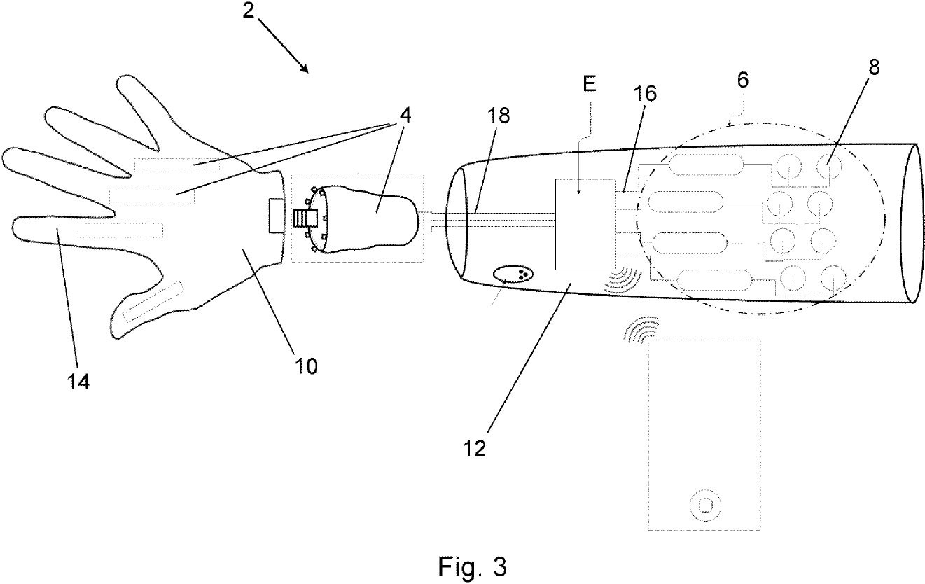

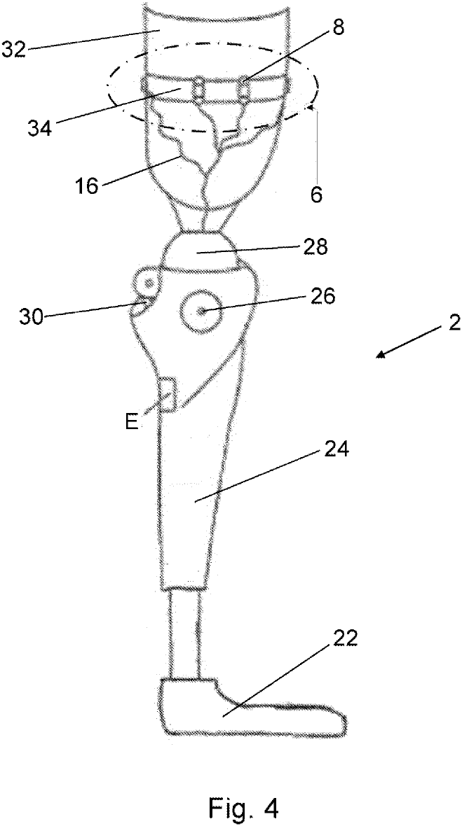

FIG. 2 shows a schematic flowchart. The prosthesis device 2 to be controlled, which is not shown in FIG. 2 but in FIGS. 3 and 4, has a sensor assembly 6 with eight electrode pairs 8, which can be seen from the eight signals indicated on the left. These signals are the result of a movement and/or a plurality of muscle contractions of a prosthesis wearer. These are routed to the electronic data processing device E where they are evaluated, i.e. possibly amplified or filtered and in particular compared to body signal patterns stored in the electronic data processing device E. In the embodiment shown in FIG. 2, two sets, S1 and S2, are stored in the electronic data processing device E. In each set, three body signal patterns X, Y and Z are stored. In the first set S1, the prosthesis movements 1 to 3 are assigned to the body signal patterns X, Y, Z. In the second set S2, the same body signal patterns X, Y, Z are assigned the prosthesis movements 4 to 6, which differ from the prosthesis movements 1 to 3.

It is possible that, as in FIG. 2, all electrode pairs 8 detect signals and send them to the electronic data processing device E. However, it is also possible that for certain body signal patterns, not all electrode pairs 8 will detect a signal.

If the electronic data processing device E has detected, for example, that the body signal pattern X is present, the electronic data processing device E then checks which of the two sets S1 or S2 is selected. The selection was made in advance, for example, by issuing an assigned trigger signal T.

If the first set S1 is selected, the prosthesis movement 1 is performed based on the detected body signal pattern X. If the second set S2 is selected, the prosthesis movement 4 is performed based on the detected body signal pattern X.

The electronic data processing device E then preferably generates a control signal that codes for the prosthesis movement to be performed and passes this control signal to the at least one drive 4 of the prosthesis device 2. The at least one drive 4 is then operated depending on the control signal in such a way that the selected prosthesis movement is performed, in this case 1 or 4, depending on which set S1 or S2 was selected.

FIG. 3 shows the schematic representation of an embodiment of the actuatable prosthesis device 2 of an upper extremity. Along its longitudinal axis, the prosthesis device 2 is partially shown in the form of an exploded view. The prosthesis device 2 is designed as a prosthesis forearm and has a plurality of drives 4, a sensor assembly 6 with a plurality of electrode pairs 8, and an electronic data processing device E.

The right-hand drive 4 shown in FIG. 3 is used to rotate a prosthetic hand 10 of the prosthesis device 2 relative to a forearm socket 12 of the prosthesis device 2 about the longitudinal axis of the forearm socket 12. In addition, the prosthetic hand 10 has a plurality of additional drives 4 within the prosthetic hand 10, which are used to actuate the prosthetic fingers 14 of the prosthetic hand 10.

The sensor assembly 6 comprises four electrode pairs 8. These are designed, for example, as individual electrode pairs 8, each of which can be attached to the skin of a prosthesis wearer. According to another embodiment, the electrode pairs 8 are applied to a prosthesis liner, not shown, for example, adhesively bonded or connected or formed integrally with the liner. According to another embodiment, the electrode pairs 8 are arranged on an inside of the forearm socket 12.

The electrode pairs 8 are each connected to the electronic data processing device E via sensor cables 16. The sensor cables 16 are used to route the signals detected by the electrodes to the electronic data processing device E.

The signals are evaluated in the electronic data processing device E, in particular to determine whether and to which body signal pattern the received signals correspond. Using the detected body signal pattern and the selected set, a prosthesis movement to be performed is then determined. A control signal coding for this prosthesis movement to be performed is then routed to the drives 4 by means of the drive cables 18.

In the embodiment shown in FIG. 3, only one of the schematically shown drive cables 18 is used for the drive 4 shown. The other drive cables 18 are routed through this or are connected to additional cables and supply the other drives 4 in the prosthetic hand 10 to actuate the finger limbs 14.

The electronic data processing device E in this case is also wirelessly connected, for example by radio, to an input device 20. This input device 20 can be used, for example, to display signals received from the sensor assembly 6. In addition, it is preferably possible to change the sets and/or trigger signals stored in the electronic data processing device E. For example, the type and number of the body signal patterns and/or prosthesis movements can be changed.

The input device 20 in this case is a smartphone, in particular of the prosthesis wearer. The wearer can use it to view the detected signals of the sensor assembly 6. In addition, it is preferable to display a target value, e.g. a target intensity, of the respective signal to the wearer. This enables them to tell whether the quality of their execution is satisfactory. If this is not the case, they can either practice or else the target values are adjusted to the result actually achieved.

FIG. 4 shows another embodiment of the actuatable prosthesis device 2, here in the form of a prosthetic leg for a thigh-amputee. The prosthesis device 2 has a sensor assembly 6 with a plurality of, e.g. six electrode pairs 8, of which only four are visible in FIG. 4.

The prosthesis device 2 comprises a prosthetic foot 22 and a lower leg section 24. On the lower leg section 24, a prosthetic knee joint 26 is positioned, which has a joint upper part 28. A damper 30 or actuator is assigned to the prosthetic knee joint 26. A prosthesis socket is arranged on the joint upper part 28, but for the sake of clarity this is not shown in FIG. 4. Instead, the limb stump 32 below it belonging to a prosthesis wearer is indicated.

The prosthetic knee joint 26 may also have a drive, not illustrated, that actuates a flexion or extension movement of the prosthesis device 2. Control signals from the electronic data processing unit E are routed to this drive 4 via drive cables 18, also not shown.

The sensor assembly 6 has a flexible, in particular elastic strap 34, on which the electrode pairs 8 are arranged. This is arranged around the limb stump 34 so that the electrode pairs 8 are in contact with it and can acquire signals accordingly. Sensor cables 16 run from each of the electrode pairs 8 to the electronic data processing device E.

List of reference signs

| | |
|---|---|
| 2 | actuatable prosthesis device |
| 4 | drive |
| 6 | sensor assembly |
| 8 | electrode pair |
| 10 | prosthetic hand |
| 12 | forearm socket |
| 14 | prosthetic finger |
| 16 | sensor cable |
| 18 | drive cable |
| 20 | input device |
| 22 | prosthetic foot |
| 24 | lower leg section |
| 26 | prosthetic knee joint |
| 28 | joint upper part |
| 30 | damper |
| 32 | limb stump |
| 34 | strap |

1. An actuatable prosthesis device, having:
at least one drive, an electronic data processing device, and at least one sensor assembly for arrangement on a body part of a prosthesis wearer;
wherein the at least one sensor assembly is designed to detect body signal patterns and the electronic data processing device is designed to actuate the at least one drive depending on a detected body signal pattern in such a way that the prosthesis device carries out a prosthesis movement assigned to the body signal pattern;
wherein at least two sets of body signal patterns and prosthesis movements assigned to each body signal pattern are stored on the electronic data processing device, and at least one trigger signal which switches over between the sets is assigned to the sets.

2. The actuatable prosthesis device of claim 1, wherein at least one body signal pattern contained in the sets is identical, and wherein the at least one identical body signal pattern contained in the sets is assigned different prosthesis movements.

3. The actuatable prosthesis device of claim 1, wherein each set contains the identical body signal patterns.

4. The actuatable prosthesis device of claim 1, wherein in a plurality of sets , preferably in every set , one or more body signal patterns is each assigned the same prosthesis movements.

5. The actuatable prosthesis device of claim 1, wherein the at least one trigger signal is a fixed body signal that differs from the body signal patterns stored in the sets.

6. The actuatable prosthesis device of claim 1, wherein each set comprises at least four body signal patterns, in particular at least six body signal patterns.

7. The actuatable prosthesis device of claim 1, wherein the sensor assembly has a plurality of electrode pairs for detecting the body signal patterns , and in particular one electrode pair for detecting the at least one trigger signal.

8. The actuatable prosthesis device of claim 1, wherein the sets can be changed using an input device.

9. An electronic data processing device for an actuatable prosthesis device of claim 1, wherein at least two sets of body signal patterns and prosthesis movements assigned to each body signal pattern are stored on the electronic data processing device, and the sets are assigned at least one trigger signal which switches over between the sets .

10. A method for actuating an actuatable prosthesis device of claim 1, having the steps:
a) selecting a set to be applied from the at least two sets stored in the electronic data processing device by issuing the at least one trigger signal,
b) detecting a body signal pattern of the prosthesis wearer using the at least one sensor assembly, and
c) actuating the prosthesis device by means of the at least one drive depending on the detected body signal pattern and the selected set , so that the prosthesis device performs the prosthesis movement assigned to the detected body signal pattern in the selected set.

11. The method of claim 10, wherein step c) also comprises the following steps:
c1) routing a sensor signal coding for the detected body signal pattern from the sensor assembly to the electronic data processing device,
c2) evaluating the sensor signal in the electronic data processing device,
c3) determining a prosthesis movement to be performed, depending on the sensor signal and the selected set, and generating a control signal that codes for the prosthesis movement to be performed,
c3) routing the control signal to the at least one drive of the prosthesis device, and
c4) operating the at least one drive depending on the control signal so that the prosthesis device performs the prosthesis movement to be performed.

12. An actuatable prosthesis device comprising:
at least one drive;
an electronic data processing device designed to actuate the at least one drive; and
at least one sensor assembly for arrangement on a body part of a prosthesis wearer, the sensor assembly being designed to detect body signal patterns of the prosthesis wearer;
wherein the at least one drive is actuated by the electronic data processing device based on a body signal pattern detected by the at least one sensor such that the prosthesis device carries out a prosthesis movement assigned to the detected body signal pattern; and
wherein a plurality of sets of body signal patterns and prosthesis movements assigned to each body signal pattern are stored on the electronic data processing device, and at least one trigger signal which switches over between the sets is assigned to the sets; and
wherein the same body signal pattern is encoded for more than one prosthesis movement.

13. The actuatable prosthesis device of claim 12, wherein the at least one trigger signal is a fixed body signal that differs from the body signal patterns stored in the sets.

14. The actuatable prosthesis device of claim 12, wherein each set comprises at least four body signal patterns.

15. The actuatable prosthesis device of claim 12, wherein each set comprises at least six body signal patterns.

16. The actuatable prosthesis device of claim 12, wherein each set comprises eight body signal patterns.

17. The actuatable prosthesis device of claim 12, wherein the sensor assembly has a plurality of electrode pairs for detecting the body signal patterns.

18. The actuatable prosthesis device of claim 17, wherein the sensor assembly includes one electrode pair for detecting the at least one trigger signal.

19. The actuatable prosthesis device of claim 1, wherein the sets can be changed using an input device.

20. An actuatable prosthesis device comprising:
at least one drive;
an electronic data processing device designed to actuate the at least one drive; and
at least one sensor assembly for arrangement on a body part of a prosthesis wearer, the sensor assembly being designed to detect body signal patterns of the prosthesis wearer;
wherein the at least one drive is actuated by the electronic data processing device based on a body signal pattern detected by the at least one sensor such that the prosthesis device carries out a prosthesis movement assigned to the detected body signal pattern; and
wherein a plurality of sets of body signal patterns and prosthesis movements assigned to each body signal pattern are stored on the electronic data processing device, and at least one trigger signal detected by an electrode pair of the at least one sensor assembly which switches over between the sets is assigned to the sets; and
wherein the same body signal pattern is encoded for more than one prosthesis movement, and the sets can be changed using an input device.
